(19) United States
(12) Patent Application Publication
(10) Pub. No.: US 2005/0009069 A1
(43) Pub. Date: Jan. 13, 2005

(54) Computer software products for analyzing genotyping

(75) Inventors: Wei-Min Liu, Dublin, CA (US); Xiaojun Di, Cupertino, CA (US); Geoffrey Yang, San Jose, CA (US)

Correspondence Address: AFFYMETRIX, INC;ATTN: CHIEF IP COUNSEL, LEGAL DEPT., 3380 CENTRAL EXPRESSWAY, SANTA CLARA, CA, 95051, US

(73) Assignee: Affymetrix, INC., Santa Clara, CA (US)

(21) Appl. No.: 10/850,183

(22) Filed: May 20, 2004

Related U.S. Application Data
(63) Continuation-in-part of application No. 10/607,108, filed on Jun. 25, 2003.
(60) Provisional application No. 60/472,369, filed on May 20, 2003.
(60) Provisional application No. 60/391,870, filed on Jun. 25, 2002.

(51) Int. Cl. C12Q 1/68; G06F 19/00; G01N 33/48; G01N 33/50

(52) U.S. Cl. 435/6; 702/20

(57) ABSTRACT

In one aspect of the invention, methods, systems and computer software products are provided for analyzing genotyping data. In exemplary embodiment, genotype data are analyzed using a model based classification method.

### RELATED APPLICATIONS

This application is a continuation-in-part of U.S. application Ser. No. 10/607,108, filed on Jun. 25, 2003 which claims priority of U.S. Provisional Application Ser. No. 60/391,870, filed on Jun. 25, 2002. This application also claims priority of U.S. Provisional Application Ser. No. 60/472,369, filed on May 20, 2003. These applications are incorporated herein in their entireties for all purposes.

### BACKGROUND OF THE INVENTION

The present invention is related to genotyping methods. More specifically, the present invention is related to computerized methods and software products for genotyping.

Genotyping methods are useful in many biological applications including drug discovery. Nucleic acid microarrays have been used for genotyping a large number of SNPs (single nucleotide polymorphisms).

### SUMMARY OF THE INVENTION

In an exemplary data analysis process, the relative allele signals for probe quartets (each probe quartet contains a perfect match (PM) for each of the two SNP alleles (A, B) and a one-base central mismatch (MM) for each of the two alleles) are calculated, and then their mean of each strand is used as the feature for that strand. The intermediate result of Wilcoxon signed rank test is used to form a feature in [0, 1]. On each of the two strands, sense and anti-sense, and each of the two types, type A and B, a discrimination score is calculated. Wilcoxon's signed rank algorithm is applied on the discrimination scores for sense and anti-sense, A and B, four detection p-values are obtained. Based on the four p-values and a significant level (with default p=0.05), if any of the detection p-values in 3.1.5 gives a present call, the SNP passes the detection filter, otherwise, it fails and is excluded.

Before PAM-based classification algorithm is processed, the detection filter is applied. Individuals who fail the detection filter will be given as no call.

MPAM-based Classification Algorithm: This algorithm use modified partitioning around medoids (MPAM) to classify genotypes based on desired features extracted.

The silhouette width is a number in the interval [−1, 1]. It is a relative measure of the difference between the distance of a data point to the nearest neighbor group and the distance of the data point to other data points in the same group. The larger the silhouette width, the better the classification from the clustering point of view, (with large distance to the nearest neighbor group and small distance to other points in the same group). It is only defined when there are two or more nonempty nonoverlapping groups.

An Average Silhouette Width is calculated based on all individuals in the classification. It can be used as a quality indication of our genotype classification from the clustering point of view. The larger the average Silhouette width, the tighter the clusters, the better the classification.

If there are already a large amount of data and need only to make genotyping calls for a few new data files, models can be established based on the classification results of the large data set (as training data set), and use the models to make calls. Since the number of model parameters is much less than the number of raw data, it helps making calls fast and storing the models with small space. With the model-based approach, the likelihood of the genotype calls can also be provided.

### BRIEF DESCRIPTION OF THE DRAWINGS

The accompanying drawings, which are incorporated in and form a part of this specification, illustrate embodiments of the invention and, together with the description, serve to explain the principles of the invention:

FIG. 1 shows an exemplary process for analyzing SNP genotyping data using PAM analysis and Classification.

FIG. 2 shows a model based SNP classification.

### DETAILED DESCRIPTION OF THE INVENTION

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

I. General

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3rd Ed., W.H. Freeman Pub., New York, N.Y. and Berg et al. (2002) Biochemistry, 5th Ed., W.H. Freeman Pub., New York, N.Y., all of which are herein incorporated in their entirety by reference for all purposes.

The present invention can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos.

PCT/US99/00730 (International Publication Number WO 99/36760) and PCT/US01/04285, which are all incorporated herein by reference in their entirety for all purposes.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

Nucleic acid arrays that are useful in the present invention include those that are commercially available from Affymetrix (Santa Clara, Calif.) under the brand name GeneChip®. Example arrays are shown on the website at affymetrix.com. The present invention also contemplates many uses for polymers attached to solid substrates. These uses include gene expression monitoring, profiling, library screening, genotyping and diagnostics. Gene expression monitoring, and profiling methods can be shown in U.S. Pat. Nos. 5,800,992, 6,013,449, 6,020,135, 6,033,860, 6,040,138, 6,177,248 and 6,309,822. Genotyping and uses therefore are shown in U.S. Ser. Nos. 60/319,253, 10/013,598, and U.S. Pat. Nos. 5,856,092, 6,300,063, 5,858,659, 6,284,460, 6,361,947, 6,368,799 and 6,333,179. Other uses are embodied in U.S. Pat. Nos. 5,871,928, 5,902,723, 6,045,996, 5,541,061, and 6,197,506.

The present invention also contemplates sample preparation methods in certain preferred embodiments. Prior to or concurrent with genotyping, the genomic sample may be amplified by a variety of mechanisms, some of which may employ PCR. See, e.g., PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188, and 5,333,675, and each of which is incorporated herein by reference in their entireties for all purposes. The sample may be amplified on the array. See, for example, U.S. Pat. No. 6,300,070 and U.S. patent application Ser. No. 09/513,300, which are incorporated herein by reference.

Other suitable amplification methods include the ligase chain reaction (LCR) (e.g., Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245) and nucleic acid based sequence amplification (NABSA). (See, U.S. Pat. Nos. 5,409,818, 5,554,517, and 6,063,603, each of which is incorporated herein by reference). Other amplification methods that may be used are described in, U.S. Pat. Nos. 5,242,794, 5,494,810, 4,988,617 and in U.S. Ser. No. 09/854,317, each of which is incorporated herein by reference.

Additional methods of sample preparation and techniques for reducing the complexity of a nucleic sample are described in Dong et al., Genome Research 11, 1418 (2001), in U.S. Pat. Nos. 6,361,947, 6,391,592 and U.S. patent application Ser. Nos. 09/916,135, 09/920,491, 09/910,292, and 10/013,598. Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2nd Ed. Cold Spring Harbor, N.Y, 1989); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference.

The present invention also contemplates signal detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. patent application 60/364,731 and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. patent application 60/364,731 and in PCT Application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, e.g. Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001).

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention may have preferred embodiments that include methods for providing genetic information over networks such as the Internet as shown in U.S. patent application Ser. Nos. 10/063,559, 60/349,546, 60/376,003, 60/394,574, 60/403,381.

II. Glossary

The following terms are intended to have the following general meanings as there used herein.

Nucleic acids according to the present invention may include any polymer or oligomer of pyrimidine and purine bases, preferably cytosine (C), thymine (T), and uracil (U), and adenine (A) and guanine (G), respectively. See Albert L. Lehninger, PRINCIPLES OF BIOCHEMISTRY, at 793-800 (Worth Pub. 1982). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally occurring sources or may be artificially or synthetically produced. In addition, the nucleic acids may be deoxyribonucleic acid (DNA) or ribonucleic acid (RNA), or a mixture thereof, and may exist permanently or transitionally in single-stranded or double-stranded form, including homoduplex, heteroduplex, and hybrid states.

An “oligonucleotide” or “polynucleotide” is a nucleic acid ranging from at least 2, preferable at least 8, and more preferably at least 20 nucleotides in length or a compound that specifically hybridizes to a polynucleotide. Polynucleotides of the present invention include sequences of deoxyribonucleic acid (DNA) or ribonucleic acid (RNA), which may be isolated from natural sources, recombinantly produced or artificially synthesized and mimetics thereof. A further example of a polynucleotide of the present invention may be peptide nucleic acid (PNA) in which the constituent bases are joined by peptides bonds rather than phosphodiester linkage, as described in Nielsen et al., Science 254:1497-1500 (1991), Nielsen Curr. Opin. Biotechnol., 10:71-75 (1999). The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

An “array” is an intentionally created collection of molecules which can be prepared either synthetically or biosynthetically. The molecules in the array can be identical or different from each other. The array can assume a variety of formats, e.g., libraries of soluble molecules; libraries of compounds tethered to resin beads, silica chips, or other solid supports.

Nucleic acid library or array is an intentionally created collection of nucleic acids which can be prepared either synthetically or biosynthetically in a variety of different formats (e.g., libraries of soluble molecules; and libraries of oligonucleotides tethered to resin beads, silica chips, or other solid supports). Additionally, the term “array” is meant to include those libraries of nucleic acids which can be prepared by spotting nucleic acids of essentially any length (e.g., from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleotide sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

“Solid support”, “support”, and “substrate” are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations.

Combinatorial Synthesis Strategy: A combinatorial synthesis strategy is an ordered strategy for parallel synthesis of diverse polymer sequences by sequential addition of reagents which may be represented by a reactant matrix and a switch matrix, the product of which is a product matrix. A reactant matrix is a 1 column by m row matrix of the building blocks to be added. The switch matrix is all or a subset of the binary numbers, preferably ordered, between 1 and m arranged in columns. A “binary strategy” is one in which at least two successive steps illuminate a portion, often half, of a region of interest on the substrate. In a binary synthesis strategy, all possible compounds which can be formed from an ordered set of reactants are formed. In most preferred embodiments, binary synthesis refers to a synthesis strategy which also factors a previous addition step. For example, a strategy in which a switch matrix for a masking strategy halves regions that were previously illuminated, illuminating about half of the previously illuminated region and protecting the remaining half (while also protecting about half of previously protected regions and illuminating about half of previously protected regions). It will be recognized that binary rounds may be interspersed with non-binary rounds and that only a portion of a substrate may be subjected to a binary scheme. A combinatorial “masking” strategy is a synthesis which uses light or other spatially selective deprotecting or activating agents to remove protecting groups from materials for addition of other materials such as amino acids.

Monomer: refers to any member of the set of molecules that can be joined together to form an oligomer or polymer. The set of monomers useful in the present invention includes, but is not restricted to, for the example of (poly)peptide synthesis, the set of L-amino acids, D-amino acids, or synthetic amino acids. As used herein, “monomer” refers to any member of a basis set for synthesis of an oligomer. For example, dimers of L-amino acids form a basis set of 400 “monomers” for synthesis of polypeptides. Different basis sets of monomers may be used at successive steps in the synthesis of a polymer. The term “monomer” also refers to a chemical subunit that can be combined with a different chemical subunit to form a compound larger than either subunit alone.

Biopolymer or biological polymer: is intended to mean repeating units of biological or chemical moieties. Representative biopolymers include, but are not limited to, nucleic acids, oligonucleotides, amino acids, proteins, peptides, hormones, oligosaccharides, lipids, glycolipids, lipopolysaccharides, phospholipids, synthetic analogues of the foregoing, including, but not limited to, inverted nucleotides, peptide nucleic acids, Meta-DNA, and combinations of the above. “Biopolymer synthesis” is intended to encompass the synthetic production, both organic and inorganic, of a biopolymer.

Related to a bioploymer is a “biomonomer” which is intended to mean a single unit of biopolymer, or a single unit which is not part of a biopolymer. Thus, for example, a nucleotide is a biomonomer within an oligonucleotide biopolymer, and an amino acid is a biomonomer within a protein or peptide biopolymer; avidin, biotin, antibodies, antibody fragments, etc., for example, are also biomonomers. Initiation Biomonomer: or “initiator biomonomer” is meant to indicate the first biomonomer which is covalently attached via reactive nucleophiles to the surface of the polymer, or the first biomonomer which is attached to a linker or spacer arm attached to the polymer, the linker or spacer arm being attached to the polymer via reactive nucleophiles.

Complementary or substantially complementary: Refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference.

The term “hybridization” refers to the process in which two single-stranded polynucleotides bind non-covalently to form a stable double-stranded polynucleotide. The term “hybridization” may also refer to triple-stranded hybridization. The resulting (usually) double-stranded polynucleotide is a “hybrid.” The proportion of the population of polynucleotides that forms stable hybrids is referred to herein as the “degree of hybridization”.

Hybridization conditions will typically include salt concentrations of less than about 1M, more usually less than about 500 mM and less than about 200 mM. Hybridization temperatures can be as low as 5° C., but are typically greater than 22° C., more typically greater than about 3Q° C., and preferably in excess of about 37° C. Hybridizations are usually performed under stringent conditions, i.e. conditions under which a probe will hybridize to its target subsequence. Stringent conditions are sequence-dependent and are different in different circumstances. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone. Generally, stringent conditions are selected to be about 5° C. lower than the thermal melting point ^{TM} fro the specific sequence at s defined ionic strength and pH. The Tm is the temperature (under defined ionic strength, pH and nucleic acid composition) at which 50% of the probes complementary to the target sequence hybridize to the target sequence at equilibrium.

Typically, stringent conditions include salt concentration of at least 0.01 M to no more than 1 M Na ion concentration (or other salts) at a pH 7.0 to 8.3 and a temperature of at least 25° C. For example, conditions of 5×SSPE (750 mM NaCl, 50 mM NaPhosphate, 5 mM EDTA, pH 7.4) and a temperature of 25-30° C. are suitable for allele-specific probe hybridizations. For stringent conditions, see for example, Sambrook, Fritsche and Maniatis. “Molecular Cloning A laboratory Manual” 2nd Ed. Cold Spring Harbor Press (1989) and Anderson “Nucleic Acid Hybridization” 1st Ed., BIOS Scientific Publishers Limited (1999), which are hereby incorporated by reference in its entirety for all purposes above.

Hybridization probes are nucleic acids (such as oligonucleotides) capable of binding in a base-specific manner to a complementary strand of nucleic acid. Such probes include peptide nucleic acids, as described in Nielsen et al., Science 254:1497-1500 (1991), Nielsen Curr. Opin. Biotechnol., 10:71-75 (1999) and other nucleic acid analogs and nucleic acid mimetics. See U.S. Pat. No. 6,156,501 filed Apr. 3, 1996.

Hybridizing specifically to: refers to the binding, duplexing, or hybridizing of a molecule substantially to or only to a particular nucleotide sequence or sequences under stringent conditions when that sequence is present in a complex mixture (e.g., total cellular) DNA or RNA.

Probe: A probe is a molecule that can be recognized by a particular target. In some embodiments, a probe can be surface immobilized. Examples of probes that can be investigated by this invention include, but are not restricted to, agonists and antagonists for cell membrane receptors, toxins and venoms, viral epitopes, hormones (e.g., opioid peptides, steroids, etc.), hormone receptors, peptides, enzymes, enzyme substrates, cofactors, drugs, lectins, sugars, oligonucleotides, nucleic acids, oligosaccharides, proteins, and monoclonal antibodies.

Target: A molecule that has an affinity for a given probe. Targets may be naturally-occurring or man-made molecules. Also, they can be employed in their unaltered state or as aggregates with other species. Targets may be attached, covalently or noncovalently, to a binding member, either directly or via a specific binding substance. Examples of targets which can be employed by this invention include, but are not restricted to, antibodies, cell membrane receptors, monoclonal antibodies and antisera reactive with specific antigenic determinants (such as on viruses, cells or other materials), drugs, oligonucleotides, nucleic acids, peptides, cofactors, lectins, sugars, polysaccharides, cells, cellular membranes, and organelles. Targets are sometimes referred to in the art as anti-probes. As the term targets is used herein, no difference in meaning is intended. A “Probe Target Pair” is formed when two macromolecules have combined through molecular recognition to form a complex.

Effective amount refers to an amount sufficient to induce a desired result.

mRNA or mRNA transcripts: as used herein, include, but not limited to pre-mRNA transcript(s), transcript processing intermediates, mature mRNA(s) ready for translation and transcripts of the gene or genes, or nucleic acids derived from the mRNA transcript(s). Transcript processing may include splicing, editing and degradation. As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, a cRNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, mRNA derived samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

A fragment, segment, or DNA segment refers to a portion of a larger DNA polynucleotide or DNA. A polynucleotide, for example, can be broken up, or fragmented into, a plurality of segments. Various methods of fragmenting nucleic acid are well known in the art. These methods may be, for example, either chemical or physical in nature. Chemical fragmentation may include partial degradation with a DNase; partial depurination with acid; the use of restriction enzymes; intron-encoded endonucleases; DNA-based cleavage methods, such as triplex and hybrid formation methods, that rely on the specific hybridization of a nucleic acid segment to localize a cleavage agent to a specific location in the nucleic acid molecule; or other enzymes or compounds which cleave DNA at known or unknown locations. Physical fragmentation methods may involve subjecting the DNA to a high shear rate. High shear rates may be produced, for example, by moving DNA through a chamber or channel with pits or spikes, or forcing the DNA sample through a restricted size flow passage, e.g., an aperture having a cross sectional dimension in the micron or submicron scale. Other physical methods include sonication and nebulization. Combinations of physical and chemical fragmentation methods may likewise be employed such as fragmentation by heat and ion-mediated hydrolysis. See for example, Sambrook et al., “Molecular Cloning: A Laboratory Manual,” 3rd Ed. Cold Spring Harbor Laboratory Press, Cold Spring Harbor, N.Y. (2001) (“Sambrook et al.) which is incorporated herein by reference for all purposes. These methods can be optimized to digest a nucleic acid into fragments of a selected size range. Useful size ranges may be from 100, 200, 400, 700 or 1000 to 500, 800, 1500, 2000, 4000 or 10,000 base pairs. However, larger size ranges such as 4000, 10,000 or 20,000 to 10,000, 20,000 or 500,000 base pairs may also be useful.

Polymorphism refers to the occurrence of two or more genetically determined alternative sequences or alleles in a population. A polymorphic marker or site is the locus at which divergence occurs. Preferred markers have at least two alleles, each occurring at frequency of greater than 1%, and more preferably greater than 10% or 20% of a selected population. A polymorphism may comprise one or more base changes, an insertion, a repeat, or a deletion. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wildtype form. Diploid organisms may be homozygous or heterozygous for allelic forms. A diallelic polymorphism has two forms. A triallelic polymorphism has three forms. Single nucleotide polymorphisms (SNPs) are included in polymorphisms.

Single nucleotide polymorphism (SNPs) are positions at which two alternative bases occur at appreciable frequency (>1%) in the human population, and are the most common type of human genetic variation. The site is usually preceded by and followed by highly conserved sequences of the allele (e.g., sequences that vary in less than 1/100 or 1/1000 members of the populations). A single nucleotide polymorphism usually arises due to substitution of one nucleotide for another at the polymorphic site. A transition is the replacement of one purine by another purine or one pyrimidine by another pyrimidine. A transversion is the replacement of a purine by a pyrimidine or vice versa. Single nucleotide polymorphisms can also arise from a deletion of a nucleotide or an insertion of a nucleotide relative to a reference allele.

Genotyping refers to the determination of the genetic information an individual carries at one or more positions in the genome. For example, genotyping may comprise the determination of which allele or alleles an individual carries for a single SNP or the determination of which allele or alleles an individual carries for a plurality of SNPs. A genotype may be the identity of the alleles present in an individual at one or more polymorphic sites.

III. SNP Genotyping Using Microarrays

The computerized methods and computer software products of the invention are particularly useful for analyzing SNP genotyping data obtained using microarrays. For the purpose of simplifying the description of the invention, the methods and computer software products of the invention will be described using exemplary embodiments in context of SNP genotyping using microarrays. However, one of skill in the art would appreciate that the scope of the invention is not limited to SNP genotyping using microarrays. Rather, the methods and computer software products of the invention are useful for analyzing a wide variety of data including genotyping (SNP or other genotypes) data obtained using different methods (such as using oligonucleotide probes immobilized on beads or optical fibers).

Applications of microarrays for SNP genotyping has been described in, e.g., a number of U.S. Patents and patent applications, including U.S. Pat. Nos. 6,300,063 6,361,947, U.S. patent application Ser. Nos. 09/916,135, 09/766,212, 10/264,945, 10/442,021, 10/321,741, 10/316,517, and 10/316,629, all incorporated herein by reference for all purposes.

Briefly, in exemplary embodiments, a DNA sample is processed to prepare the target and the processed DNA sample is hybridized with a genotyping high density oligonucleotide probe array.

In an exemplary target preparation process, total genomic DNA (250 ng) is incubated with 20 units of EcoRI, BglII or XbaI restriction endonuclease (New England Biolabs) at 37° C. for 4 hrs. Following heat inactivation at 75° C. for 20 min, the digested DNA is incubated with 0.25 uM adaptors and DNA ligase (NEB) in standard ligation buffer (NEB) at 16° C. for 4 hrs. The sample is incubated at 95° C. for 5 min to inactivate the enzyme. Target amplification is performed with ligated DNA and 0.5 uM primer in PCR Buffer II (Perkin Elmer) with 2.5 mM MgCl2, 250 uM dNTPs and 50 units of Taq polymerase (Perkin Elmer). Cycling is conducted as follows: 95° C./10 min followed by 20 cycles of 95° C./10s, 58° C./15 sec, 72° C./15 sec, followed by 25 cycles of 95° C./20 sec, 55° C./15 sec, 72° C./15 sec. Final extension is performed at 72° C. for 7 minutes. The amplification products are concentrated with a YM30 column (Microcon) centrifuged at 14,000 rfc for 6 min. Column is washed twice with 400 ul H2O, respun at 14,000 rfc, inverted and the sample recovered in a clean tube by centrifuging at 3000 rfc for 3 min. The sample is digested with 0.045 units DNase (Affymetrix) and 0.5 units calf intestinal phosphatase (Gibco) in RE Buffer #4 (NEB) at 37° C. for 30 minutes. Enzymes are inactivated at 95° C. for 15 min. Samples are labeled with 15-20 units Terminal deoxytransferase (Promega), 18 uM biotinylated ddATP (NEN) in TdT buffer (Promega) at 37° C. for 4 hrs. Following heat inactivation at 95° C. for 10 min, samples are injected into microarray cartridges and hybridized overnight following manufacturer's directions (Affymetrix). Microarrays are washed in a fluidics station (Affymetrix) using 0.6×SSPET, followed by a three-step staining protocol. First the arrays are incubated with 10 ug/ml streptavidin (Pierce), followed by a wash with 6×SSPET, followed by 10 ug/ml biotinylated anti-streptavidin (Vector Lab), 10 ug/ml streptavidin-phycoerythrrin conjugate (Molecular Probes) and a final wash of 6×SSPET. Microarrays are scanned according to manufacturer's directions (Affymetrix).

In one exemplary embodiment, for each SNP, four probes (25-mers) are synthesized, spanning seven positions along both strands of the SNP-containing sequence, with the SNP position in the center, (position zero) as well as at −4, −2, −1, +1, +3, +4. Probes may be synthesized for both sense and antisense strands. Four probes are synthesized for each of the 7 positions: a perfect match (PM) for each of the two SNP alleles (A, B) and a one-base central mismatch (MM) for each of the two alleles. These four probes are referred to as a probe quartet.

IV. Genotyping Algorithm

The following sections describe various algorithms for genotyping. Some of the algorithms are also described in U.S. Provisional Application Ser. No 60/423,073, which is incorporated herein by reference.

A. Feature Extraction Algorithms

1 Mathematical Details of Rank-Based Algorithms.

The signed rank test applies to two paired data sets:

\overline{x}=(x₁, x₂, . . . ,xₙ) and \overline{y}=(y₁,y₂, . . . ,yₙ), It can test the null hypothesis:
H₀:median(xᵢ−yᵢ)=0
versus the alternative hypothesis
H₁:median(xᵢ−yᵢ)>0
Typically, the genotyping algorithm uses the one-sided test. For the one-sided test, if the null hypothesis is true, the p-value should be close to 0.5. When the alternative hypothesis is true, the p-value should be close to 0. When
median(xᵢ−yᵢ)<0
is true, the p-value should be close to 1. This property makes the one-sided test useful for both absolute and comparative calls. As a standard procedure of signed rank test, the exemplary algorithm first calculates the differences of all pairs of data:
dᵢ=xᵢ−yᵢ A1
If all differences are zero, the algorithm outputs 0.5 as the one-sided p-value. If some of the differences are zero, the algorithm excludes them from further analysis and use only the nonzero differences for further analysis. The remaining nonzero difference is denoted as dᵢ(i1, . . . ,n). Their absolute values are:
aᵢ=|dᵢ| A2
and sort aᵢ in ascending order. If all aᵢ's are different from each other, they are ranked with integers from 1 to n, and assigned the original signs to these ranks to form the signed ranks. Let us denote the ranks by rᵢ and the signed rank of dᵢ by sᵢ. If there are ties among the absolute values of differences aᵢ, all differences in a tie group are assigned a rank equal to the average of the integer ranks. For example, if five nonzero differences are
d₁=2, d₂=1, d₃=−2, d₄=0.5, d₅=0.5
then their ranks are
r₁=4.5, r₂=3, r₃=4.5, r₄=1.5, r₅=1.5
and their signed ranks are
s₁=4.5, s₂=3, s₃=−4.5, s₄=1.5, s₅=1.5
The sum of positive signed ranks is:

$$S = {\sum }_{i = 1}^{n} u ( {s}_{i} ) {s}_{i} \qquad A3$$

where u(sᵢ)=1 if sᵢ>0, u(sᵢ)=0 if sᵢ<0. For our example,
S=s₁+s₂+s₄+s₅=10.5 A4
If xᵢ and yᵢ are symmetrically distributed around a common median, S should be close to n(n+1)/4; if median(xᵢ) is significantly larger than median(yᵢ), S should be close to its maximal value n(n+1)/2; if median(xᵢ) is significantly smaller than median(yᵢ), S should be close to its minimal value 0. The one-sided p-value can better describe these different situations. When n is small (e.g., n<11), the algorithm can assign signs randomly to ranks rᵢ(i=1, . . . ,n), calculate the sum of positive ranks and denote this sum by Sⱼ(j=1, . . . ,2ⁿ). In many statistical definitions, the p-value of S is defined as

$$p ( S ) = \frac{1}{{2}^{n}} {\sum }_{j = 1}^{{2}^{n}} u ( {S}_{j} \geq S ) \qquad A5$$

where u(Sⱼ≧S)=1 if Sⱼ≧S, otherwise 0. An alternative definition is employed in preferred exemplary embodiments:

$$p ( S ) = \frac{1}{{2}^{n}} {\sum }_{j = 1}^{{2}^{n}} ( u ( {S}_{j} > S ) + 0.5 u ( {S}_{j} = S ) \qquad A6$$

For comparative calls, definition (A6) may work better because it has the property

$$p ( \frac{n ( n + 1 )}{2} - S ) + p ( S ) = 1 \qquad A7$$

In our above example, the random signed ranks and the sum of positive ranks S′ are list in Table 1.

TABLE 1
Random Signed Ranks for p-value Evaluation

| Index j | s′_1 | s′_2 | s′_3 | s′_4 | s′_5 | S_j |
|---|---|---|---|---|---|---|
| 1 | −1.5 | −1.5 | −3 | −4.5 | −4.5 | 0 |
| 2 | 1.5 | −1.5 | −3 | −4.5 | −4.5 | 1.5 |
| 3 | −1.5 | 1.5 | −3 | −4.5 | −4.5 | 1.5 |
| 4 | −1.5 | −1.5 | 3 | −4.5 | −4.5 | 3 |
| 5 | −1.5 | −1.5 | −3 | 4.5 | −4.5 | 4.5 |
| 6 | −1.5 | −1.5 | −3 | −4.5 | 4.5 | 4.5 |
| 7 | 1.5 | 1.5 | −3 | −4.5 | −4.5 | 3 |
| 8 | 1.5 | −1.5 | 3 | −4.5 | −4.5 | 4.5 |
| 9 | 1.5 | −1.5 | −3 | 4.5 | −4.5 | 6 |
| 10 | 1.5 | −1.5 | −3 | −4.5 | 4.5 | 6 |
| 11 | −1.5 | 1.5 | 3 | −4.5 | −4.5 | 4.5 |
| 12 | −1.5 | 1.5 | −3 | 4.5 | −4.5 | 6 |
| 13 | −1.5 | 1.5 | −3 | −4.5 | 4.5 | 6 |
| 14 | −1.5 | −1.5 | 3 | 4.5 | −4.5 | 7.5 |
| 15 | −1.5 | −1.5 | 3 | −4.5 | 4.5 | 7.5 |
| 16 | −1.5 | −1.5 | −3 | 4.5 | 4.5 | 9 |
| 17 | 1.5 | 1.5 | 3 | −4.5 | −4.5 | 6 |
| 18 | 1.5 | 1.5 | −3 | 4.5 | −4.5 | 7.5 |
| 19 | 1.5 | 1.5 | −3 | −4.5 | 4.5 | 7.5 |
| 20 | 1.5 | −1.5 | 3 | 4.5 | −4.5 | 9 |
| 21 | 1.5 | −1.5 | 3 | −4.5 | 4.5 | 9 |
| 22 | 1.5 | −1.5 | −3 | 4.5 | 4.5 | 10.5 |
| 23 | −1.5 | 1.5 | −3 | 4.5 | 4.5 | 10.5 |
| 24 | −1.5 | 1.5 | 3 | −4.5 | 4.5 | 9 |
| 25 | −1.5 | 1.5 | 3 | 4.5 | −4.5 | 9 |
| 26 | −1.5 | −1.5 | 3 | 4.5 | 4.5 | 12 |
| 27 | 1.5 | 1.5 | 3 | 4.5 | −4.5 | 10.5 |
| 28 | 1.5 | 1.5 | 3 | −4.5 | 4.5 | 10.5 |
| 29 | 5 | 1.5 | −3 | 4.5 | 4.5 | 12 |
| 30 | 1.5 | −1.5 | 3 | 4.5 | 4.5 | 13.5 |
| 31 | −1.5 | 1.5 | 3 | 4.5 | 4.5 | 13.5 |
| 32 | 1.5 | 1.5 | 3 | 4.5 | 4.5 | 15 |

In our example, if definition (A5) is used, p(10.5)=9/32=0.28125, and if one interchanges xᵢ and yᵢ, p(15−10.5)=27/32=0.84375, their sum is 1.125. However, if definition A6) is used, p(10.5)=(5+0.5·4)/32=0.21875, and if one interchanges xᵢ and yᵢ, p(15−10.5)=(23+0.5·4)/32=0.78125, their sum is 1. When n is large, e.g., n>11, one can use asymptotic approximation. The statistic

$${S}^{′} = \frac{S - n ( n + 1 ) / 4}{\sqrt{n ( n + 1 ) ( 2 n + 1 ) / 24 - {\sum }_{k = 1}^{t} {b}_{k} ( {b}_{k}^{2} - 1 ) / 48}} \qquad A8$$

is considered to have a standard normal distribution with mean 0 and variance 1, where t is the number of tie groups, bₖ is the number of ties in the k-th tie group.
2. Simplified Relative Allele Signal.

Let PMA(i) be the i-th perfect match intensity of type A, MMA(i) be the i-th mismatch intensity of type A, PMB(i) be the i-th perfect match intensity of type B, MMB(i) be the i-th mismatch intensity of type B. It is defined:
MM(i)=(MMA(i)+MMB(i))/2,
A(i)=max (PMA(i)−MM(i),0),
B(i)=max (PMB(i)−MM(i),0) A11
The simplified relative allele signal is defined to be

$$R = \frac{{\sum }_{i} A ( i )}{{\sum }_{i} ( A ( i ) + B ( i ) )} \qquad A12$$

3. Median of Relative Allele Signal.

Let n be the number of probe pairs for a type (either A or B), let
d(i)=A(i)+B)i), i=1,2, . . . ,n
One can define \overline{r}=(r(1), r(2), . . . ,r(n)) as the discrimination score vector, where

$$r ( i ) = \{ \begin{matrix}A ( i ) / d ( i ) , & if d ( i ) > 0 \\ - 2 , & otherwise\end{matrix} \qquad A13$$

Remove all negative elements from vector \overline{r}, the remaining vector is
ti \overline{r}′=(r′(1),r′(2), . . . ,r′(m)) A14
Where {r′(1),r′(2), . . . ,r′(m)} is a subset of {r(1),r(2), . . . ,r(n)}. The median of relative allele signal is defined as the median of vector \overline{r}′.
4. Mean of Relative Allele Signal.

The mean of relative allele signal is defined as the mean of vector \overline{r}′ as defined in (A14).

5. Relative Sum of Signed Ranks.

The relative sum of signed ranks is another feature can be used for genotyping algorithms. In Wilcoxon's signed rank test, the sum of positive signed ranks, S, for a vector of n components may be calculated. The relative sum of signed ranks is defined to be

$$r = \frac{2 S}{n ( n + 1 )} \qquad A15$$

which is a quantity in the interval [0, 1]. Specifically, the vector is formed with components
v(i)=(PMA(i)−MMA(i)−(PMB(i)−MMB(i)),
v(n1+i)=c(PMA(i)−PMB(i)),
For i=1,2, . . . ,n₁ where n₁ is the common size of vectors PMA, MMA, PMB and MMB, n=2n₁, and c is parameter with default value 1.
6. Discrimination Scores.

Let n be the number of probe pairs for a type (either A or B), we define \overline{r}=(r(1),r(2), . . . ,r(n)) as the discrimination score vector for a specific strand, where
r(i)=(PM(i)−MM(i))/(PM(i)+PM(i)), i=1, . . . ,n A17
7. Detection p-Values.

By applying Wilcoxon signed rank test on the following hypothesis
H₀:median(\overline{r})=Γ
versus the alternative hypothesis
H₁:median(\overline{r})>Γ
where Γ is the threshold with default value of 0.015, p-values are obtained.

B. Detection Filter Algorithm.

Let p₁,p₂,p₃, p₄ be the four p-values obtained as in A7, we can define

p=min {p₁,p₂,p₃,p₄} B1

as the detection p-value, if
p>=α B2
the individual will be excluded for classification, α is the significant level with default value of 0.05.

C. Classification Algorithms

1. PAM and MPAM

For all quantities, the method of partition around medoids (PAM, Kaufman L. and P. J. Rousseeuw, Finding Groups in Data: An Introduction to Cluster Analysis. John Wiley & Sons, New York, pp. 68-123, 1990; Struyf, A. Hubert, M. and P. J. Rousseeuw, Integrating robust clustering techniques in S-Plus. Computational Statistics & Data Analysis, 26, 17-37, 1997) may be used for classification. The algorithm may consider two features, one for sense and the other for antisense. The algorithm can classify the data in the 2-dimensional feature space with PAM. PAM is a robust classification method using the distance (or dissimilarity) matrix.

1.1. Modified Partitioning Around Medoids.

The partitioning around medoids is a robust classification method based on a dissimilarity matrix. It can well classify most SNPs, but sometimes it can be improved. In one aspect of the invention, a modified partitioning around medoids (MPAM) is provided. The method includes PAM as a special case when the parameter λ=0. MPAM can be considered as unsupervised clustering because MPAM itself does not assign the genotypes. However, immediately after MPAM, the genotypes can be assigned based on the median coordinates of clusters. Moreover, the number of clusters (2 or 3) is pre-determined.

Let n be the number of distinct points, and we consider the problem of classifying them into k groups (1<k<n). In the case of genotyping, we may have k=1, 2, or 3. Classification is done for k=2 and 3. If the results of classification for k=2 and 3 are of low quality, the data are considered as from one group. Let d(xᵢ,xⱼ) be the Euclidian distance between points xᵢ and xⱼ. PAM minimizes the objective function

$$f = {\sum }_{i = 1}^{n} {min}_{j = 1 , … , k} d ( {x}_{i} , {m}_{j} )$$

for a subset (mₗ, . . . ,mₖ) of (x₁, . . . , xₙ), and m₁, . . . ,mₖ are called the medoids of groups G₁, . . . ,Gₖ. PAM minimizes the sum of distances of all points to the nearest medoids without consideration of the distances between groups. When there are significantly more points in a group than those in another group, PAM tends to separate the large group into two small groups to reduce the total sum of distances of all points to the nearest medoids. MPAM penalizes the small between-group distances. MPAM minimizes the new objective function

$$g = f - λ {\sum }_{j = 1}^{k} {D}_{j} where {D}_{j} = {min}_{{x}_{a} ∈ {G}_{j} , {x}_{b} ∉ {G}_{j}} ( d ( {x}_{a} , {x}_{b} ) )$$

is the smallest distance of group Gⱼ to any point in other groups. The non-negative coefficient λ can adjust the penalty of small between-group distances.
1.2. Features to Form the Feature Space.

Mean of type discrimination scores for two strands, sense and anti-sense.

Median of type discrimination scores for two strands, sense and anti-sense.

Relative sum of signed rank statistics for two strands, sense and anti-sense.

2. Predetermined 2-d Regions.

For the relative sum of signed ranks, in addition to PAM, the algorithm may also use predetermined 2-d regions to classify the data. Let x and y be the relative sum of signed ranks for sense and antisense strands. The region of type AA is defined by
x+y>β or (x>y₁& y>y₂) or (x>y₂ & y>y₁) C2
The region of type BB is defined by
x+y<−β or (x<−y₁ & y<−y₂) or (x<−y₂ & y<−y₁) C3
The remaining region is of type AB.

D. Classification Quality Algorithims

1. Average Silhouette Width.

Average silhouette width can be used to quantify the quality of classification. The silhouette width is a number in the interval [1, −1]. It is a relative measure of the difference between the distance of a data point to the nearest neighbor group and the distance of the data point to other data points in the same group. The larger the silhouette width, the better the classification from the clustering point of view, larger distance to the nearest neighbor group and smaller distance to other points in the same group. It is defined only when there are two or more nonempty non-overlapping groups.

Let i be a data point in group G. If i is the only point in G, its silhouette value is defined to be s(i)=0. If there are more than one point in group G, s(i) is defined in terms of a(i) and b(i). Here a(i) is its average distance to other points in group G:

$$a ( i ) = \frac{1}{\vert G \vert - 1} {\sum }_{j ∈ G , j \neq i}^{} d ( i , j )$$

where |G| is the number of points in group G. Let the distance of i to another group C be

$$d ( i , C ) = \frac{1}{\vert C \vert} {\sum }_{j ∈ C}^{} d ( i , j ) \qquad D2$$

The distance of i to the nearest neighbor group is

$$b ( i ) = {min}_{C \neq G} d ( i , C ) \qquad D3$$

The silhouette value s(i) of the data point i is defined to be

$$s ( i ) = \frac{b ( i ) - a ( i )}{max ( b ( i ) , a ( i ) )} \qquad D4$$

If s(i) is close to 1, i is well classified in group G, i.e., its distance to other points in the same group is much smaller than its distance to the nearest neighbor group. If s(i) is close to 0, i has similar distances to other data points in group G and to the nearest neighbor group. If s(i) is close to −1, i is badly classified from the clustering point of view because its distance to other points in the same group is much larger than its distance to the nearest neighbor group. One exemplary embodiment defines the average silhouette width for the whole data set as

$$s = \frac{1}{n} {\sum }_{i = 1}^{n} s ( i ) \qquad D5$$

It can be used as a quality indication of our genotype classification from the clustering point of view.
2. Separation of Groups.

Another measure of quality is the separation of groups. The algorithm first takes medians of features of every group (AA, AB or BB). The separation is defined to be the minimum of the distance between AB and AA medians and the distance between the AB and BB medians. Sense separation and antisense separation are calculated separately.

3. _{χ}²—Test for Hardy-Weinberg Equilibrium.

In some embodiments, a _{χ}² test for the Hardy-Weinberg equilibrium (Hartl, D. L. and Jones, E. W., Genetics: Analysis of Genes and Genomes, 5^{th} edition. Jones and Bartlett, Boston, 2001) is included in the computerized methods and software products. t the observed genotype frequencies be ƒ_{AA}, ƒ_{AB} and ƒ_{BB}. The observed allele frequencies are

$$\begin{matrix}{f}_{A} = {f}_{AA} + 0.5 {f}_{AB} , {f}_{B} = {f}_{BB} + 0.5 {f}_{AB} \\ We form & D6 \\ x = \frac{{( {f}_{A}^{2} - {f}_{AA} )}^{2}}{{f}_{A}^{2}} + \frac{{( {f}_{B}^{2} - {f}_{BB} )}^{2}}{{f}_{B}^{2}} + \frac{{( {f}_{A} {f}_{B} - {f}_{AB} )}^{2}}{2 {f}_{A} {f}_{B}} \\ The p \text{-} value is & \text{D7} \\ {p}_{HW} = 1 - {cdf}_{{χ}^{2}} ( x , df ) & \text{D8}\end{matrix}$$

where the degree of freedom dƒ=1, and cdf_{χ}_{²} is the cumulative distribution function of the _{χ}² distribution.

E. Model-Based Call Algorithm

MPAM takes much time to find the global optimized solution. If there are already a large amount of data and need only to make genotyping calls for a few new data files, models can be established based on the classification results of the large data set (as training data set), and use the models to make calls. Since the number of model parameters is much less than the number of raw data, it helps making calls fast and storing the models with small space. With the model-based approach, we can also provide the likelihood of the genotype calls.

1. Multivariate Normal Models.

Assume, we find m (m is 2 or 3) clusters with a classification method, e.g., modified partitioning around medoids (MPAM), and they have good average silhouette widths and good separations. Let the n points in the feature space (we use the 2-dimensional feature space as an example, but it can be 1-dimensional or higher dimensional) be

$${x}_{ij} = ( \begin{matrix}{x}_{ij}^{( 1 )} \\ {x}_{ij}^{( 2 )}\end{matrix} ) , i = 1 , … , m , j = 1 , … , {n}_{i} , {\sum }_{i = 1}^{m} {n}_{i} = n \qquad E1$$

When m=3 and nᵢ>1, (i=1,2,3), we define the classification as good. Based on this classification, we can find the centroids

$${\overline{x}}_{ij} = {\sum }_{j = 1}^{{n}_{i}} \frac{{x}_{ij}}{{n}_{i}} \qquad E2$$

and the sample covariance matrices

$${S}_{i} = \frac{( {X}_{i} - {\overline{x}}_{i} {\vec{e}}^{′} ) {( {X}_{i} - {\overline{x}}_{i} {\vec{e}}^{′} )}^{′}}{{n}_{i} - 1} \qquad E3$$

where Xᵢ=(\overline{x}ᵢₗ, . . . ,\overline{x}ᵢₙ_{ᵢ}). and \overline{e}′ is a vector whose components are all 1. Sᵢ is always positive semidefinite. For good models, if the determinant of Sᵢ is very close to 0, we can increase the diagonal elements by a tiny positive number so that it becomes positive definite. We can form the quadratic discriminant (Johnson, R. A. and Wichem, D. W., Applied Multivariate Statistical Analysis (fourth edition). Prentice-Hall, Upper Saddle, N.J., 1998).

$${d}_{i}^{Q} ( \vec{y} ) = - \frac{1}{2} ln \vert {S}_{i} \vert - \frac{1}{2} {( \vec{y} - {\overline{x}}_{i} )}^{′} {S}_{i}^{- 1} ( \vec{y} - {\overline{x}}_{i} ) + ln {p}_{i} \qquad E4$$

where pᵢ is the prior probability. We use three different choices, (1) pᵢ=1/m, which is equivalent to not using pᵢ, (2) pᵢ=nᵢ/n, and (3), pᵢ=ƒᵢ, where ƒᵢ is the Hardy-Weinberg frequency calculated from the observed allele frequency. We can allocate y to group k if dₖ^{Q}(\overline{y}) is the largest of d₁^{Q}(\overline{y}), . . . , dₘ^{Q}(\overline{y}). Similarly, we can also form the linear discriminate
dᵢ(\overline{y})=\overline{x}ᵢS⁻¹pooled (\overline{y}−0.5\overline{x}ᵢ)+1n pᵢ E5
Here the pooled covariance matrix is

$${S}_{pooled} = {\sum }_{i = 1}^{m} ( {n}_{i} - 1 ) {S}_{i} / {\sum }_{i = 1}^{m} ( {n}_{i} - m ) \qquad E6$$

Algorithm can allocate y to group k if dₖ(\overline{y}) is the largest of d₁(\overline{y}), . . . , dₘ(\overline{y}).
2. Estimate Models with the Average Model or Locally Weighted Regression Smoothing.

When the classification of a marker does not have good separation or good average silhouette width, we use the average of good models for that marker. If the classification of a marker has good separation and good average silhouette width, but m=2, or m=3 and some nᵢ=1, we can estimate the unknown parameters by the locally weighted regression smoothing (Hastie, T. and Tibshirani, R., Generalized Additive Models. Chapman and Hall, London, 1990).

Let the known parameters in a model be \overline{p}₀, find K nearest good models with corresponding parameters \overline{p}ᵢ(i=1, . . . ,K). Let the largest distance be

$$D = {max}_{i = 1 , ⋯ , K} d ( {{p}^{->}}_{0} , {{p}^{->}}_{1} ) \qquad E7$$

where d(\overline{p}₀,\overline{p}ᵢ) is the distance between parameter vectors \overline{p}₀ and \overline{p}ᵢ. For fast computation, we use the 1-distance. Define the weight function

$$W ( u ) = \{ \begin{matrix}{( 1 - {u}^{3} )}^{3} , & if u ∈ [ 0 , 1 ) \\ 0 , & otherwise\end{matrix} \qquad E8$$

Calculate the weights
wᵢ=W(d(\overline{p}₀,\overline{p}ᵢ)/D) E9
The unknown parameters \overline{q}₀ is estimated with the weighted average

$${\vec{q}}_{0} = \frac{{\sum }_{i = 1}^{K} {w}_{i} {\vec{q}}_{i}}{{\sum }_{k = 1}^{K} {W}_{i}} \qquad E10$$

The locally weighted regression smoothing method can also be used to good models when the number of points are too few to give dependable estimation of covariance matrices.
3. Call Quality.

Exemplary methods and software products report the probabilities of observations belonging to the three genotypes. They can also be called likelihoods. The sum of these three numbers is equal to 1. For the quadratic discriminants, they are defined to be

$${L}_{i}^{Q} ( \vec{y} ) = \frac{exp ( {d}_{i}^{Q} ( \vec{y} ) )}{{\sum }_{j = 1}^{3} exp ( {d}_{j} ( \vec{y} ) )} \qquad E11$$

For the linear discriminant, we define

$${L}_{i} ( \vec{y} ) = \frac{exp ( {d}_{i} ( \vec{y} ) )}{{\sum }_{j = 1}^{3} exp ( {d}_{j} ( \vec{y} ) )} \qquad E12$$

The call is given to the genotype with the largest probability. The larger the largest probability, the better the quality of the call under the given model. For example, if the probabilities for AA, AB and BB are respectively 0.0001, 0.0002 and 0.9997, we may consider it as a very good BB call for the particular model. If these numbers are 0.1, 0.4 and 0.5, we also call it BB, but it might also be AB type. If these numbers are 0.2, 0.4 and 0.4, we give “no call”, but from these numbers, we know it is either AB or BB. Please note that these probabilities are calculated using the model parameters and they do not form a quality measure of the model itself.
4. Robust Model.

In another aspect of the invention, a robust model modified from the classical multivariate normal model with equal prior probabilities and the covariance matrices equal to the same multiple of the identity matrix is provided. Under these assumptions, the probability of a point in a group is consistent with its proximity to the group center, and we can use Fisher's linear discriminants. we use sample medians to estimate the group centers. Let's consider k groups with multivariate normal distributions N(xᵢ,σ²I) (i=1, . . . ,k). The linear discriminant dᵢ(y)=xᵢ(y−0.5xᵢ). The point y is classified to group j if j=arg max,(dᵢ(y)). The variance σ² can be estimated with median(r²)/(2ln2), where r² is the squared distance of a classified point to the corresponding distribution center. We divide the models into three tiers based on the classification quality and accept or adjust the models accordingly. The model of a SNP belongs to the first tier if it has good three-group classification, i.e., there are at least two points in every group and the average silhouette width and separation are large enough. We accept the first tier models without adjustment. If the three-group classification has large enough average silhouette width and separation but a group has only one point, we categorize the model as in the second tier. If the average silhouette width or the separation of the three-group classification is not large enough, but the two-group classification has large enough average silhouette width and separation, we also rank the two-group model as in the second tier. For models in the second tier, we use the locally weighted regression smoothing to estimate the center of distribution for the group with only one or zero point based on the models in the first tier. All other models are categorized as in the third tier, which includes the situation that there is really only one group and both 2- or 3-group classifications are of low quality. We use the average of the first tier models as the model for a SNP in the third tier of classification. The locally weighted regression smoothing can be described as follows. Let the known good parameters, e.g., the centers of two groups in a second tier model be p₀, find K nearest first tier models with corresponding parameters pᵢ(i=1, . . . ,K). Let the largest distance be L=max_{i=1, . . . ,K}d(p₀,pᵢ) where d(p₀,pᵢ) is the distance between parameter vectors p₀ and pᵢ. For fast computation, we use the 1-distance. The weight function w(u)=(1−u³ )³, if u ε[0,1]; and w(u)=0, otherwise. We calculate wᵢ=w(d(p₀,pᵢ)/L). The other parameters q₀, e.g., the center of the group with 0 or 1 point, is estimated as

$${q}_{0} = {\sum }_{i = 1}^{K} {q}_{i} {w}_{i} / {\sum }_{i = 1}^{K} {w}_{i}$$

Since male has a single X chromosome and Y chromosome, the genotype of a SNP on the X or Y chromosomes for a male sample can only be homozygous. For male samples, we should use only two-group classification for SNPs on the X or Y chromosomes. To reach high accuracy, we implemented the following post-call filters. The probability of a type i call by using robust model is proportional to exp(dᵢ(y)/σ²).
We denote the largest discriminant as max(dᵢ(y)) and the second largest discriminant as second(dᵢ(y)). Their rescaled difference c=[max(dᵢ(y))−second(dᵢ(y))]/(σ²ln10) is the logarithm with base 10 of the probability ratio and can be used as a confidence measure.

IV. Computerized Methods, Systems and Software Products for Genotyping

The algorithms described above outline the method steps for performing various analytical methods. The methods are typically performed by computers. In some embodiments, a computerized method for building a model for analyzing genotyping data include the steps of imputing probe intensities from multiple samples, wherein the probes are designed to interrogate a SNP; performing a feature extraction on the probe intensities; performing a partition around medioids (PAM) analysis or MPAM and classification; and building a SNP model. The genotyping data from multiple samples are typically a training data set. Once the models are built based upon the training data set, they can be used to analyze genotyping data to determine genotypes. The method steps (algorithm) are described in great detail in the above section. In some preferred embodiments, average silhouette width or other measures are calculated for quantifying the quality of the classification. The feature extraction step typically includes analyzing the intensities using a rank-based analysis, such as analyzing the relative sum of signed ranks. Optionally, the feature extraction may include applying a detection filter. The feature extract step may include estimating a relative allele signal (RAS), which can be used to build models. Exemplorary models are described in above sections. Suitable models include a multivariate normal model which includes a sample covariance matrices. The models are very useful for analyzing genotyping date from individual samples. A typically method includes imputing in probe intensities from a sample, wherein the probes are designed to interrogate a SNP; performing a feature extraction on the probe intensities; performing a model based classification. Preferred methods may include calculating the classification quality.

In one aspect of the invention, computer software products and computer systems are provided to perform the methods (algorithms) described above.

Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Computer systems of the invention typically include at least one CPU coupled to a memory. The systems are configured to store and/or execute the computerized methods discribed above. Basic computational biology methods are described in, e.g. Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001).

V. Exemple

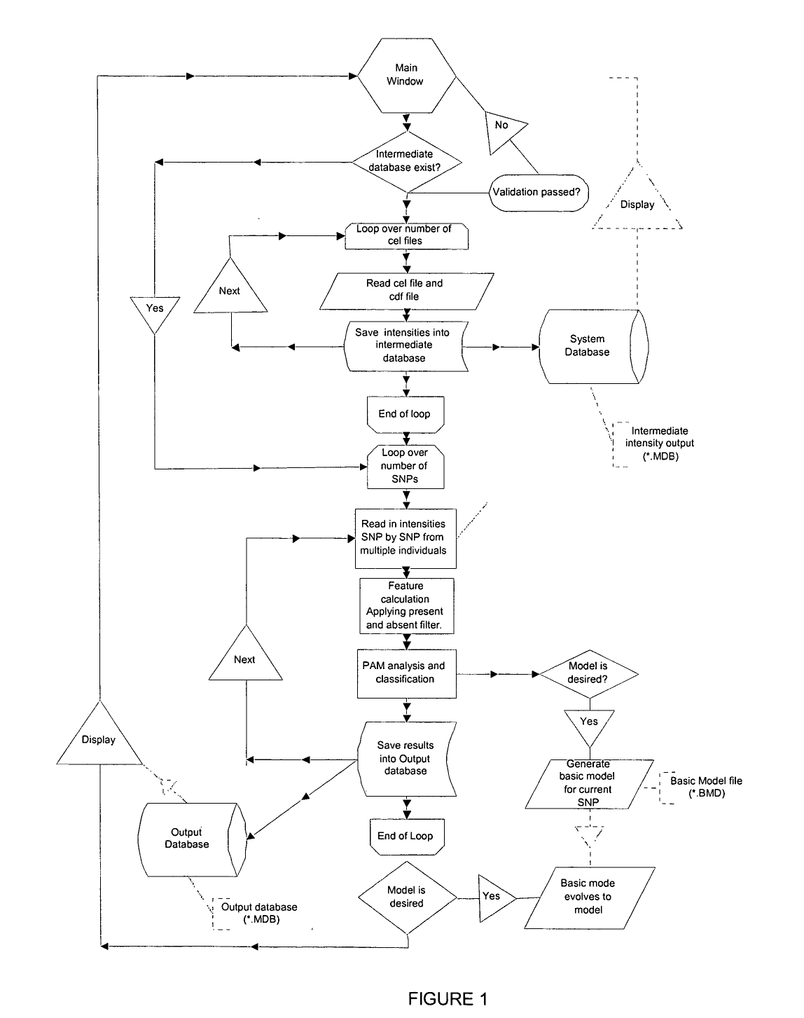

FIG. 1 shows an exemplary computerized process for generating models for genotyping analysis. This process was implemented in computer software products including the Affymetrix® Genotyping Tools and is also discribed in Affymetrix® Genotyping Tools User's Guide (Affymetrix, Santa Clara, Calif.). Genotyping data typically are probe intensities. In this exemple, the probe intensities are stored in data files such as the Affymetrix standard .cel file. The intensities are read and stored in a optional system database for ease of further analyzsis. Intensities from multiple samples (individuals) are analyzed per SNP. Feature extraction algorithms described above are employed to obtain RAS data for PAM analysis and classification. If a model is desirable, a basic model is generated. The basic model may be evolved into a model that is used for genotyping analysis.

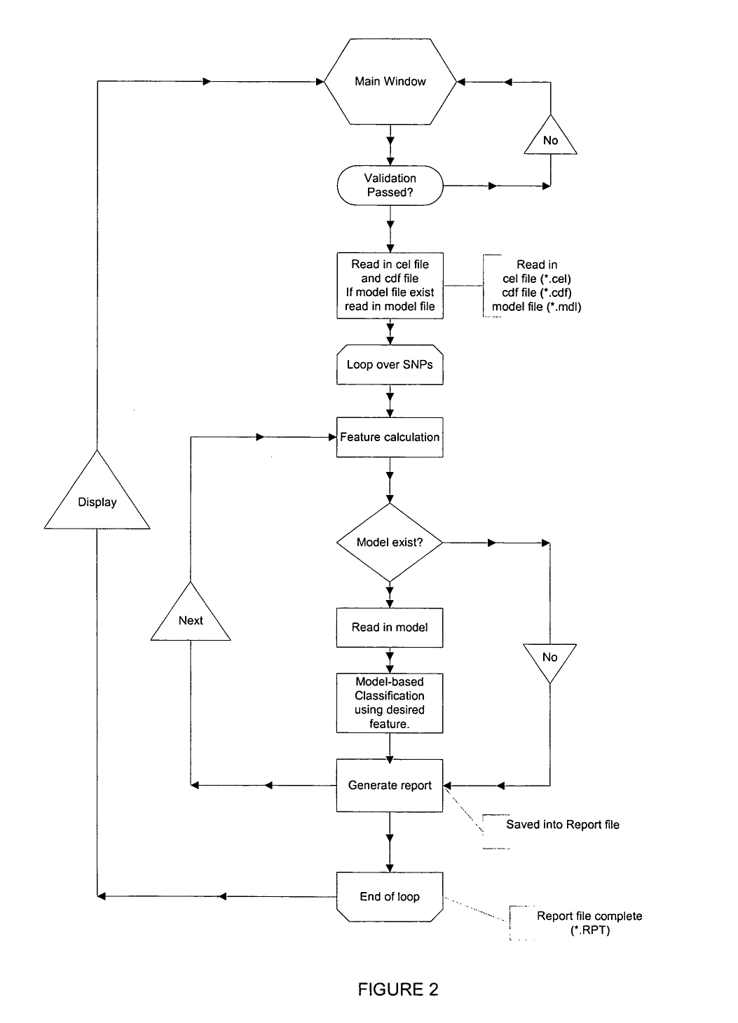

FIG. 2 shows a process for analyzing a SNP using the genotyping model. This process was also implemented in computer software products including the Affymetrix® Genotyping Tools and is also discribed in Affymetrix® Genotyping Tools User's Guide (Affymetrix, Santa Clara, Calif.). In this process, probe intensities are inputted and analyzed for feature extraction. Model based classification are then performed to make genotyping calls.

It is to be understood that the above description is intended to be illustrative and not restrictive. Many variations of the invention will be apparent to those of skill in the art upon reviewing the above description. All cited references, including patent and non-patent literature, are incorporated herein by reference in their entireties for all purposes.

1. A computer software product for building a model for analyzing genotyping data comprising a computer readable medium having computer-executable instructions for performing the logic steps comprising:
Imputing probe intensities from multiple samples, wherein the probes are designed to interrogate a SNP;
Performing a feature extraction on the probe intensities;
Performing a partition around medioids (PAM) analysis and classification; and
Building a SNP model.

2. The computer software product of claim 1 further comprising calculating average silhouette width for quantifying the quality of the classification.

3. The computer software product of claim 1 wherein the feature extraction comprises analyzing the intensities using a rank-based analysis.

4. The computer software product of claim 3 wherein the feature extraction comprises analyzing the relative sum of signed ranks.

5. The computer software product of claim 4 wherein the feature extraction comprises applying a detection filter.

6. The computer software product of claim 5 wherein the feature extraction comprises estimating a relative allele signal (RAS).

7. The computer software product of claim 6 wherein the model is a multivariate normal model.

8. The computer software product of claim 7 wherein the multivariate normal model comprises a sample covariance matrices.

9. A computer software product for analyzing genotyping data comprising a computer readable medium having computer-executable instructions for performing the logic steps comprising:
Imputing in probe intensities from a sample, wherein the probes are designed to interrogate a SNP;
Performing a feature extraction on the probe intensities;
Performing a model based classification.

10. The computer software product of claim 9 wherein the feature extraction comprises analyzing the intensities using a rank-based analysis.

11. The computer software product of claim 9 wherein the feature extraction comprises analyzing the relative sum of signed ranks.

12. The computer software product of claim 11 wherein the feature extraction comprises applying a detection filter.

13. The computer software product of claim 12 wherein the feature extraction comprises estimating a relative allele signal (RAS).

14. The computer software product of claim 9 wherein the model is a multivariate normal model.

15. The computer software product of claim 9 wherein the multivariate normal model comprises a sample covariance matrices.

16. The computer software product of claim 9 further comprising calculating the classification quality.
